(19) United States
(12) Patent Application Publication
(10) Pub. No.: US 2010/0247374 A1
(43) Pub. Date: Sep. 30, 2010

(54) DEVICE FOR MAKING A LIQUID SAFE BY ULTRAVIOLET RADIATION AT THE DISPENSING POINT AND METHOD FOR MAKING A LIQUID SAFE

(75) Inventor: Xavier Pellet, Meylan (FR)

Correspondence Address: OLIFF & BERRIDGE, PLC, P.O. BOX 320850, ALEXANDRIA, VA, 22320-4850, US

(73) Assignee: RC-LUX, Meylan (FR)

(21) Appl. No.: 12/743,660

(22) PCT Filed: Nov. 26, 2008
(86) PCT No.: PCT/FR2008/001645

§ 371 Date: May 19, 2010

(30) Foreign Application Priority Data
Nov. 27, 2007 (FR) 0708279

(51) Int. Cl. A61L 2/10; G05D 7/00

(52) U.S. Cl. 422/24; 422/107

(57) ABSTRACT

The invention relates to a device for making a liquid safe, that comprises a sterilizer including an ultraviolet lamp, and a sensor for measuring the ultraviolet radiation and connected to an electronic board. An actuator is connected to the electronic board driving the opening of an electrovalve in order to dispense or stop dispensing the liquid, said electrovalve being provided upstream from the sterilizer relative to the liquid flow direction. The electronic board controls the opening of the electrovalve when the sensor measures an ultraviolet amount higher than a predetermined threshold for a given amount of liquid. The device is further fitted with a flow rate adjustment means adapted to the predetermined threshold. The invention also relates to a method for implementing said device.

### BACKGROUND OF THE INVENTION

The invention relates to a device for making a liquid safe at the dispensing point comprising:
a sterilizer comprising an ultraviolet lamp, an inlet port and an outlet port of said liquid,
supply means of the liquid connected to the inlet port of the sterilizer,
distribution means of the liquid, in direct pressure flow, comprising a first end connected to the outlet port of the sterilizer and a second end in the form of a neck,
an actuator connected to a printed circuit card controlling lighting of the ultraviolet lamp and opening of a solenoid valve for distribution of the liquid or closing of said solenoid valve to stop distribution of the liquid,
and a sensor measuring the ultraviolet radiation connected to the printed circuit card to control closing of the solenoid valve when a signal corresponding to a quantity of ultraviolet radiation lower than a predefined threshold is emitted by the sensor.

### STATE OF THE ART

Liquid purifying devices by ultraviolet radiation exist that treat the liquid at the dispensing point (generally a neck where the liquid is recovered) so as to make the latter safe in accordance with a standard. What is defined by safe liquid is a liquid that has been irradiated by a sufficient quantity of ultraviolet radiation to eradicate micro-organisms present in the liquid. The optimal wavelength to eradicate micro-organisms (viruses, bacteria, algae, etc.) corresponds to ultraviolet C radiation (254 nm) that is able to penetrate into the heart of the DNA to disturb the metabolism of the cells so as to destroy the latter. An UV ray threshold corresponds to a cumulated quantity of UV energy, depending of the radiation power and of the irradiation time, that the living micro-organisms have to absorb to be destroyed.

For example purposes, the following table presents the quantities, expressed in millijoules/cm², necessary to eradicate 99.9% of micro-organisms:

| | Quantity | |
|---|---|---|
| Bacteria | | |
| Bacillus Anthracis | 8.5 | mJ/cm² |
| E. Coli | 10.5 | mJ/cm² |
| Legionella Pneumophilia | 6.9 | mJ/cm² |
| Pseudomonas Aeruginosa | 10 | mJ/cm² |
| Salmonella Enteridis | 9 | mJ/cm² |
| Streptococcus Faecalis | 10 | mJ/cm² |
| Algae | | |
| Chlorella Vulgaris | 22 | mJ/cm² |
| Protozoa | | |
| Cryptosporidium | 16 | mJ/cm² |
| Viruses | | |
| Hepatitis | 8 | mJ/cm² |

The document U.S. Pat. No. 6,909,101 describes one such device as illustrated in FIG. 1. The treatment device is a water purifying apparatus comprising a sterilizer 1 comprising an ultraviolet lamp 2, an inlet port 3 and outlet port 4 of a liquid that can be water. Inlet port 3 is connected to water supply means 5. Outlet port 4 is connected to a first end 6b of water distribution means 6. A second end of distribution means 6 is in the form of a neck 6a acting as dispensing point. When the device is installed on a support 7, sterilizer 1 and supply means 5 are located underneath this support 7 and distribution means 6 pass through support 7. An actuator 8 is situated at the level of the junction between a top part of support 7 and distribution means 6. Such an actuator 8 can be an infrared sensor. Actuator 8 is connected to a printed circuit card 9 controlling opening of a solenoid valve 10 situated upstream from sterilizer 1 with respect to the direction of flow of the water (illustrated by an arrow in FIG. 1), upstream being everything that is located before the inlet port 3 of sterilizer and downstream being everything that is located after the outlet port 4 of the sterilizer. A pressure detector 11 is placed between solenoid valve 10 and water inlet port 3 of sterilizer 1. When this pressure detector 11 detects a pressure greater than a certain threshold, pressure detector 11 informs printed circuit card 9 of this and the latter lights ultraviolet lamp 2 of sterilizer 1. The lamp is therefore only lit when it is sure that the latter will receive water in order to avoid damaging the latter. The water flowing through the sterilizer is treated by irradiation of the ultraviolet rays emitted by lamp 2. Such a device does not enable it to be guaranteed that each drop of water output from neck 6a of distribution means 6 has been made safe when passing through sterilizer 1. Such a device will in particular be used in an intermittent operating mode such as a kitchen sink tap. The lighting time of lamp 2 not being instantaneous, a few seconds to a few minutes to reach a sufficient quantity of ultraviolet rays, the water distributed is not made safe during this lapse of time.

The document U.S. Pat. No. 5,611,918 describes a water treatment device limiting the problem of the foregoing device. The treatment device comprises a sterilizer comprising an inlet port and an outlet port of the liquid, a water flow detector placed upstream from the sterilizer with respect to the direction of the water flow, connected to a printed circuit card, and an ultraviolet lamp also connected to the printed circuit card. The printed circuit card comprises two operating modes, when no water flow is detected by the flow detector, the lamp is supplied by the printed circuit card at low intensity. On the contrary, when a water flow is detected, the lamp is supplied with a high intensity. At low intensity, the filaments of the lamp remain weakly excited which enables them to be rapidly re-intensified when a water flow is detected. Although this device limits the number of non-treated water drops, it does not enable it to be guaranteed that all the drops of liquid passing through the device are treated. This device further generates considerable energy losses. It is difficult to install this device at the level of a potable water dispensing point. In the latter case, the sterilizer has to operate in intermittent manner, i.e. when the liquid is no longer distributed, the ultraviolet lamp has to be extinguished. Water in fact contains nitrates, and when the ultraviolet radiation threshold exceeds 400 mJ/cm², the nitrates are transformed into nitrites that are dangerous for health. This transformation of nitrates into nitrites can take place even if the lamp is lit at low intensity. Furthermore, if the lamp remains lit without the liquid being distributed, the temperature of the liquid is increased and development of biofilms in the distribution means is fostered, not to mention the fact that a warm water is not pleasant to consume.

The document WO2004/073754 describes a device for sterilizing a fluid at the dispensing point for a shower. The device comprises a sterilizer equipped with an ultraviolet lamp and a photosensitive sensor measuring the quantity of ultraviolet radiation emitted by the lamp. Distribution of the liquid is automatically loop-locked to a two-minute delay necessary to light the lamp, and on completion of this time delay distribution can begin. In operation, if the sensor measures a lower quantity of ultraviolet radiation than a certain threshold, distribution is stopped. Such a device is viable in the field of showers but cannot be used in the field of potable water fountains with intermittent operation, the lag time of two minutes not being able to be tolerated by a user wanting to quench his thirst.

### OBJECT OF THE INVENTION

The object of the invention consists in providing a device for making a liquid safe at the dispensing point by ultraviolet radiation guaranteeing a totally safe distribution of liquid in an intermittent operating mode.

This object is achieved by the fact that the printed circuit card comprises means for controlling opening of the solenoid valve when the sensor measures a larger quantity of ultraviolet radiation than the predefined threshold for a given flowrate, and that the device is equipped with means for adjusting the flowrate adapted to the predefined threshold.

Such a device guarantees that each drop of the liquid passing through the sterilizer is treated by a sufficient quantity of ultraviolet radiation to guarantee a safe distribution of liquid.

According to an alternative embodiment, the solenoid valve is situated upstream from the sterilizer with respect to the direction of flow of the liquid.

According to one feature of the invention, the distribution means of the liquid comprise a pipe left free and without obstacles, on outflow from the outlet port of the sterilizer up to the neck of the distribution means.

According to a development, the distribution means present an internal surface in contact with the liquid, made from biocidal materials, in particular silver-doped, or reflecting ultraviolet radiation emitted by the lamp.

According to another feature of the invention, means for accelerating lighting of the ultraviolet lamp optimize the speed of distribution of the liquid.

According to an alternative embodiment, the printed circuit card comprises auxiliary triggering means to purge the sterilizer, the distribution means and supply means.

The invention also relates to a method for using the device, characterized by the following successive steps:
a first signal is emitted to a printed circuit card by activating an actuator,
lighting of an ultraviolet lamp of a sterilizer is commanded when the printed circuit card receives the first signal,
a quantity of ultraviolet radiation emitted by the lamp is measured by means of a measurement sensor emitting a second signal to the printed circuit card until a predefined threshold is exceeded for a given flowrate,
the solenoid valve is opened on receipt of a third signal emitted by the printed circuit card when said card receives the second signal exceeding the predefined threshold,
   and the solenoid valve is closed if the quantity of ultraviolet radiation emitted by the lamp drops back below said threshold, or when the actuator is activated again.

### BRIEF DESCRIPTION OF THE DRAWINGS

Other advantages and features will become more clearly apparent from the following description of a particular embodiment of the invention given for non-restrictive example purposes only and represented in the accompanying drawings, in which:

FIG. 1 illustrates a cross-sectional view of a device according to the prior art.

FIG. 2 schematically illustrates an embodiment of the invention.

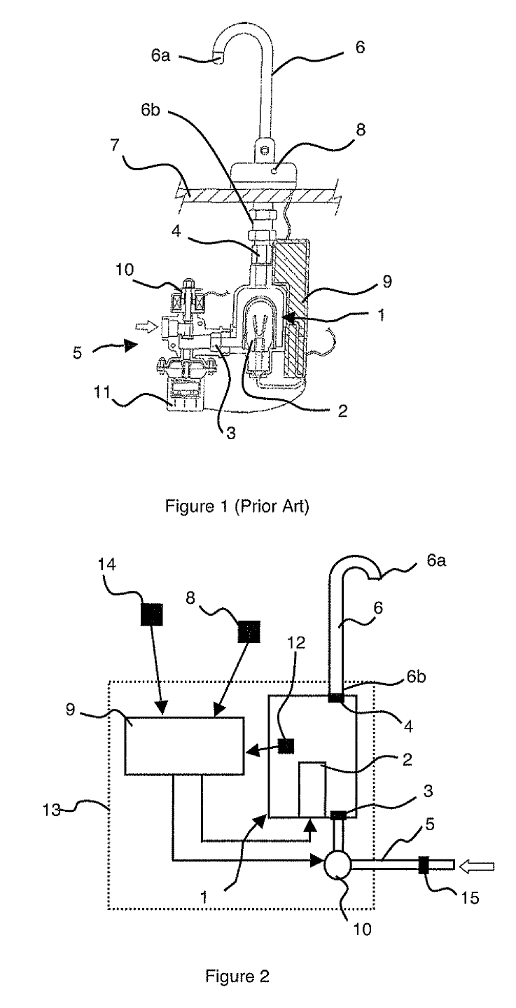

### DESCRIPTION OF A PREFERRED EMBODIMENT

The device for making a liquid safe at the dispensing point, illustrated in FIG. 2, comprises a sterilizer 1 of the liquid comprising an ultraviolet lamp 2 and a sensor 12 measuring the ultraviolet radiation emitted by lamp 2. The sensor is preferably arranged in the sterilizer facing ultraviolet lamp 2. Sterilizer 1 further comprises an inlet port 3 for inlet of the liquid, to which supply means 5 are connected, and an outlet port 4 connected to a first end 6b of distribution means 6. Distribution means 6 comprise a second end in the form of a neck 6a corresponding to the dispensing point. Distribution means 6 are in direct pressure flow, i.e. the flowrate of the liquid between inlet port 3 and distribution means 6 is the same. The liquid is not stored in a tank for example where it could be contaminated. The device comprises a printed circuit card 9 on which an actuator 8 and sensor 12 measuring the ultraviolet radiation are connected. Printed circuit card 9 controls a solenoid valve 10 and lighting of lamp 2 of sterilizer 1 according to the data from the actuator and from the sensor. The solenoid valve is preferably situated upstream from sterilizer 1 with respect to the direction of the liquid flow (indicated by an arrow in FIG. 2) and enables distribution of the liquid by opening solenoid valve 10 or stops distribution by closing solenoid valve 10 on activation of the actuator. Printed circuit card 9 comprises means for controlling opening of solenoid valve 10 when sensor 12 measures a larger quantity of ultraviolet radiation than a predefined threshold for a given liquid flowrate.

When actuator 8 is activated, a signal is transmitted to printed circuit card 9 which determines that the liquid has to be distributed. Printed circuit card 9 then commands lighting of ultraviolet lamp 2. Printed circuit card 9 receives signals, via sensor 12, corresponding to the measured quantity of ultraviolet radiation emitted by lamp 2. When measurement of the radiation considered is higher than a certain predefined threshold, printed circuit card 9 determines that sterilizer 1 is ready to treat the liquid. At this moment, printed circuit card 9 commands opening of solenoid valve 10 and the liquid passes through sterilizer 1 via inlet port 3. The liquid is then irradiated by the ultraviolet radiation emitted by lamp 2, and each drop of the liquid comes out treated via outlet port 4. During distribution of the liquid, if the quantity of ultraviolet radiation measured by the sensor drops back below the predefined threshold, printed circuit card 9 commands closing of solenoid valve 10 to stop distribution of the liquid.

The predefined threshold of the quantity of ultraviolet radiation is defined for a given liquid flowrate and corresponds to sanitary and/or medical standards enabling micro-organisms to be destroyed (for example see the above table). In order to prevent any development of micro-organisms in distribution means 6 of the liquid, the latter can comprise a pipe left free and without obstacles, on outflow from outlet port 4 of the sterilizer to neck 6a of distribution means 6.

The predefined threshold depends on the flowrate of the liquid flowing through the sterilizer. To install the device for making liquid safe on any installation and/or to be able to palliate flowrate fluctuations of the installation where the device is used due to pressure variations, the latter is equipped with flowrate adjustment means 15 adapted to the threshold. Flowrate adjustment means 15 can be in the form of a diaphragm or a self-limiting solenoid valve. It is thereby possible to guarantee a flowrate in the sterilizer corresponding to the predefined ultraviolet radiation threshold. A self-limiting solenoid valve can comprise a flow meter commanding opening and closing of the self-limiting solenoid valve to ensure a constant flowrate. According to an alternative embodiment, it is also possible to modify the predefined threshold corresponding to a quantity of ultraviolet radiation according to the flowrate. The lower the flowrate the more the corresponding threshold will be reduced. Flowrate adjustment means 15 can for example be a simple plate, a diaphragm or a self-limiting solenoid valve connected to printed circuit card 9. Printed circuit card 9 then controls the flowrate and computes the associated threshold. Flowrate adjustment means 15 enable the device to be installed without prior adjustment of the flowrate, everything being automated.

The method for making a liquid safe at the dispensing point making use of a sterilizer with ultraviolet radiation and a solenoid valve as described above comprises the following successive steps:
a first signal is emitted to a printed circuit card 9 by activating an actuator 8,
lighting of an ultraviolet lamp 2 of a sterilizer 1 is commanded when printed circuit card 9 receives the first signal,
a quantity of ultraviolet radiation emitted by lamp 2 is measured by means of a measuring sensor 12 emitting a second signal to printed circuit card 9 until a predefined threshold, guaranteeing that a liquid flowing through the sterilizer will be made safe for a given flowrate, is exceeded,
the solenoid valve is opened on receipt of a third signal emitted by printed circuit card 9 when said card 9 receives the second signal exceeding the predefined threshold.

Once the solenoid valve has been opened, the liquid is distributed to neck 6a of the device, this liquid being totally safe as each drop has been treated by a quantity of ultraviolet radiation corresponding to sanitary standards. Solenoid valve 10 is reclosed if the quantity of ultraviolet radiation emitted by the lamp drops back below said threshold (lamp malfunction, scale formation, change of nature of the water, etc.), or when the actuator is activated again to request that distribution be stopped, thereby making the liquid safe throughout distribution of the latter.

Preferably, when the actuator has been activated to request that distribution of the liquid be stopped, the solenoid valve is closed and ultraviolet lamp 2 is then extinguished after a lag time following full closure of solenoid valve 10, for example by a lag time of a few seconds so that the liquid contained in the sterilizer can be sufficiently treated so as not to pollute the sterilizer. This lag time is determined by computation using conventional methods to determine a sufficient irradiation of a liquid according to the predefined threshold, the power of the ultraviolet lamp and the volume of liquid the sterilizer can accommodate.

As ultraviolet radiation does not have remanent effects (unlike chlorine), the non-safe liquid can contaminate distribution means 6 and foster the development of a biofilm (aggregation of micro-organisms) downstream from the sterilizer, which would make it impossible to make the liquid safe at neck 6a. Any obstacle should therefore advantageously be placed upstream from sterilizer 1, and solenoid valve 10 and flowrate adjustment means 15 are therefore preferably placed upstream from the sterilizer.

Furthermore, to limit the development of biofilms in distribution means 6, the latter present an internal surface in contact with the liquid that is as smooth as possible. The surface in contact with the liquid is made from biocidal materials, for example doped with silver. Such a doping limits formation of biofilms. The ultraviolet radiation can just as well be introduced into distribution means 6 by optic fibres, for each fibre one end being directed towards ultraviolet lamp 2 in the sterilizer and the other end terminating in distribution means 6. According to a development, the internal surface reflects the ultraviolet radiation emitted by ultraviolet lamp 2, enabling the radiation to be propagated in the distribution means.

In an alternative embodiment, lamp 2 of the sterilizer can be formed by an electrode-free ultraviolet lamp to enable an unlimited number of lighting/extinguishing cycles. Such a lamp avoids inconveniences that may arise when an electrode lamp is used. A large number of lighting/extinguishing cycles does in fact damage electrode lamps resulting in numerous maintenance operations. For example, the lamp used to treat the liquid by irradiation with ultraviolet rays can be a diode lamp, an induction lamp, microwave lamp etc. Electrode lamps can naturally be used but will have to be replaced more often, moreover such lamps take a longer time to reach the sufficient quantity of ultraviolet radiation to make the liquid safe.

When the device is not used, lamp 2 can be lit regularly for a short time in time-lagged manner to create a bacteriological barrier between upstream and downstream of the sterilizer without the solenoid valve being open. This barrier enhances the safety of the liquid when the solenoid valve is situated upstream from the sterilizer. Even when the water is not flowing, bacteria are in fact mobile. The barrier prevents bacteria from migrating from upstream to downstream of the sterilizer and/or vice-versa. When solenoid valve 10 is closed, the bacteriological barrier prevents retrocontamination and development of bacteria in the sterilizer. In an alternative embodiment, printed circuit card 9 can comprise auxiliary means for triggering automatic purging to purge sterilizer 1, distribution means 6 and supply means 5. When the device has not been used for a certain time, a time setting that can be adjustable, the auxiliary triggering means of printed circuit card 9 execute a system purge cycle. This cycle is similar to use of the device by activating actuator 8 as described above. To determine whether a purge cycle is to be performed, printed circuit card 9 stores the time when the device was used for the last time, and if the elapsed time is longer than a certain threshold, printed circuit card 9 then initiates lighting of lamp 2. When the measuring sensor informs printed circuit card 9 that the lamp is emitting a sufficient quantity of ultraviolet radiation to guarantee distribution of a safe liquid, printed circuit card 9 commands opening of solenoid valve 10 to perform purging by rinsing with purified liquid, and then recloses the solenoid valve once the purging operation has been completed. In another alternative embodiment, purging is performed in two steps. On a command from printed circuit card 9, when lamp 2 emits sufficient ultraviolet radiation, a descaling and anti-biofilm product contained in a storage tank connected to the sterilizer is added automatically or on request, and rinsing is then performed with ultraviolet-purified liquid. The anti-biofilm product eliminates micro-organisms (bacteria, fungi, algae, or protozoa) that have adhered to one another to form a biofilm on an inside surface of sterilizer 1 and distribution means 6 where the liquid flows.

Supply means 5 of the liquid can comprise a filter of charcoal filter type (not shown) to improve the taste and reduce the presence of chlorine in the liquid. As the charcoal filter fosters development of micro-organisms, it should preferably be fitted upstream from the sterilizer. A purge cycle will enable the water present in the filter to be renewed to prevent proliferation of micro-organisms upstream from sterilizer 1.

According to a feature of the invention, means for accelerating lighting of ultraviolet lamp 2 optimize the speed of distribution of the liquid. The speed of distribution can be defined by the waiting time of a user between activation of actuator 8 and the moment the liquid purified by ultraviolet irradiation flows out from distribution means 6 via neck 6a. The means for accelerating can correspond to the power supplied to lamp 2. Printed circuit card 9 comprises a first supply mode of lamp 2 provided with a greater power than the reference power to irradiate the liquid and a second supply mode of lamp 2 having a power regulated to the reference power. The reference power corresponds to the supply power required for lamp 2 to emit ultraviolet radiation corresponding to the predefined threshold. Lamp 2 is therefore over-supplied until the measurement made by sensor 12 reaches the required ultraviolet threshold, and when the threshold is reached, the power is regulated by printed circuit card 9 to maintain a constant irradiation quantity.

In another alternative embodiment, the means for accelerating distribution of the liquid correspond to anticipation of lighting of lamp 2 by regular flashes which maintain the safety of the water in the sterilizer when the water is not flowing, or by a presence detector (not shown). The detector is then connected to printed circuit card 9 to control lighting of the lamp when a presence is detected by the detector. In this way when a user activates the actuator, the liquid distribution time is shorter than the heating time of lamp 2 or even instantaneous if the cumulated quantity of ultraviolet radiation of the lamp has been sufficient. Lamp 2 can then be extinguished when the detector no longer detects any movement after a certain time delay. This alternative embodiment can cooperate with one or both of the alternative embodiments described above.

According to another alternative embodiment, flowrate adjustment means 15 can be used coupled to means for accelerating lighting of the lamp so as to enable a limited flowrate of safe liquid to be ensured during the heating time of lamp 2 followed by a nominal flowrate when lamp 2 emits the quantity of ultraviolet radiation associated with the nominal flowrate.

Sterilizer 1, solenoid valve 10, and printed circuit card 9 can be integrated in a sealed housing 13 and be connected to a common power supply. If the device comprises flowrate adjustment means 15, the latter can also be integrated in the housing. The housing preferably has a protection factor of 23 (IP23). The indication IP23 is a safety standard meaning for its first digit (2) that the housing is protected against solid bodies larger than 12 mm being able to penetrate and for its second digit (3) that the housing is also protected against rain falling on the housing with an angle of 60°. Such housings can also be used without any danger near to water points of a bathroom or a kitchen. In an alternative embodiment, the housing comprises a hatch for access to the sterilizer so as to be able to change the lamp without having to dismantle the housing thereby making maintenance operations of the device easier.

Printed circuit card 9 can comprise at least one network interface (not shown) connected to a computer network. This interface is of hardwired or wireless network type and enables the data of a sterilizer to be centralized and in particular enables diagnostics to be performed. For example, it is possible to know the state of the lamp, to perform tests, to perform purging, etc. Printed circuit card 9 can be remote controlled so as to simulate manual activation of the actuator.

The device can incorporate display means 14 to indicate different states of the sterilizer. These display means are situated at the level of housing 13 or can be transferred remotely to a sink on which the device is installed enabling the user to be informed of the state of the sterilizer (quality of the liquid, lamp pre-heating, etc.).

The system maintenance data can also be communicated to the user. Need for cleaning due to fouling or scaling of the system can for example be detected by the printed circuit card and the UV measuring sensor by comparing the standard ageing slope of the lamp and the actual slope measured by the sensor.

Actuator 8 is a manual actuator enabling water distribution to be put into operation or stopped. It can for example be in the form of a switch, an infrared detector, a presence detector, a voice recognition system, or an electronic faucet not comprising a valve the external appearance whereof is that of a traditional tap, etc. For use thereof not to confuse a user, actuator 8 can be arranged on a base of distribution means 6 downstream from the sterilizer. The actuator can comprise a washer compatible with standard sink holes and, in the case where the external appearance of the latter corresponds to a traditional tap, a potentiometer connected to printed circuit card 9. An electronic faucet with a potentiometer can control the flowrate of the liquid or a mixer situated upstream from solenoid valve 10 in order to regulate the temperature of the liquid. The actuator can also incorporate the display means to indicate different states of the system to a user.

The pipe forming distribution means 6 can be in the form of a removable goose-neck for ease of cleaning if necessary.

The device and its alternative embodiments as described above can be installed at sink level to enable distribution of liquid and more particularly of water. This device can in particular be used in hospitals where the needs for safe water supply are great, as well as in countries where the local water system is not very reliable. The device enables it to be guaranteed that each drop of liquid flowing through the sterilizer is irradiated by a calibrated quantity of ultraviolet radiation.

1. Device for making a liquid safe at the dispensing point comprising:
a sterilizer comprising an ultraviolet lamp, an inlet port and an outlet port of said liquid,
supply means of the liquid connected to the inlet port of the sterilizer,
distribution means of the liquid, in direct pressure flow, comprising a first end connected to the outlet port of the sterilizer and a second end in the form of a neck,
an actuator connected to a printed circuit card controlling lighting of the ultraviolet lamp and opening of a solenoid valve for distribution of the liquid or closing of said solenoid valve to stop distribution of the liquid,
and a sensor measuring the ultraviolet radiation connected to the printed circuit card to control closing of the solenoid valve when a signal corresponding to a quantity of ultraviolet radiation lower than a predefined threshold is emitted by said sensor,
wherein said printed circuit card comprises means for controlling opening of the solenoid valve when the sensor measures a larger quantity of ultraviolet radiation than the predefined threshold for a given flowrate, and in that the device is equipped with means for adjusting the flowrate adapted to the predefined threshold.

2. Device according to claim 1, wherein said solenoid valve is situated upstream from the sterilizer with respect to the direction of flow of the liquid.

3. Device according to claim 1, wherein the distribution means of the liquid comprise a pipe left free and without any obstacles, on outflow from the outlet port of the sterilizer up to the neck of the distribution means.

4. Device according to claim 3, wherein the distribution means comprise an internal surface in contact with the liquid, made from biocidal materials in particular silver-doped, and/or reflecting the ultraviolet radiation emitted by the lamp.

5. Device according to claim 1, wherein it comprises means for accelerating lighting of the ultraviolet lamp.

6. Device according to claim 5, wherein the printed circuit card is connected to a presence detector to anticipate lighting of the ultraviolet lamp when a presence is detected by said detector.

7. Device according to claim 1, wherein the printed circuit card comprises auxiliary triggering means to purge the sterilizer, the distribution means and the supply means of the liquid.

8. Device according to claim 1, wherein the solenoid valve being closed, the device comprises a bacteriological barrier between the upstream and downstream of the sterilizer, said bacteriological barrier being generated by regular lighting of the ultraviolet radiation lamp for a short time.

9. A method for making liquid safe at the dispensing point making use of a device comprising an ultraviolet radiation sterilizer and a solenoid valve according to claim 1, comprising the following successive steps:
a first signal is emitted to a printed circuit card by activating an actuator,
lighting of an ultraviolet radiation lamp of a sterilizer is commanded when the printed circuit card receives the first signal,
a quantity of ultraviolet radiation emitted by the ultraviolet lamp is measured by means of a sensor emitting a second signal to the printed circuit card until a predefined threshold for a given flowrate is exceeded,
the solenoid valve is opened on receipt of a third signal emitted by the printed circuit card when said card receives the second signal exceeding the predefined threshold,
and the solenoid valve is closed if the quantity of ultraviolet radiation emitted by the ultraviolet lamp drops back below said threshold or when the actuator is activated again.

10. Method according to claim 9, wherein the actuator being activated again to request that distribution of liquid be stopped, the solenoid valve is closed and said ultraviolet radiation lamp is extinguished after a time lag that is a function of the predefined threshold, of the power of the ultraviolet lamp and of the volume of the sterilizer.
